(19) United States
(12) Patent Application Publication
(10) Pub. No.: US 2019/0021830 A1
(43) Pub. Date: Jan. 24, 2019

(54) METHOD FOR CLEANING DENTAL IMPLANTS AND DEVICE FOR PERFORMING THE SAME

(71) Applicant: Wen Kai KAO, Taipei City (TW)

(72) Inventor: Wen Kai KAO, Taipei City (TW)

(21) Appl. No.: 16/042,143

(22) Filed: Jul. 23, 2018

Related U.S. Application Data
(60) Provisional application No. 62/535,532, filed on Jul. 21, 2017.

(51) Int. Cl. A61C 17/00; A61C 8/00; A61L 27/32

(52) U.S. Cl. CPC A61C 17/036; A61L 2430/12; A61L 27/32; A61C 8/0013

(57) ABSTRACT

The present invention provides a method for cleaning a dental implant in a patient, comprising placing an isolation device around the dental implant to isolate the dental implant from other teeth of the patient, and air polishing the surface of the dental implant with β-tricalcium phosphate powder having a particle size equal to or less than 350 μm for a period of no more than 90 seconds.

### CROSS REFERENCE TO RELATED APPLICATIONS

This non-provisional application claims the benefit under 35 U.S.C. § 119(e) to U.S. Provisional Application No. 62/535,532, filed on Jul. 21, 2017, which is hereby expressly incorporated by reference into the present application.

### FIELD OF THE INVENTION

The present invention relates to a method for cleaning a dental implant in a patient, and a device for performing the same.

### BACKGROUND OF THE INVENTION

Dental implants have become increasingly available and affordable to patients and the use of dental implants is generally more desirable than false teeth which are temporarily adhered to the gum. However, currently available methods for cleaning a dental implant or a post thereof do not always achieve a satisfactory result and have problems with damaging of tissues. For example, glycine powder air-polishing would result in erosions of the gingival epithelium (Petersilka G et al., J Clin Periodontol 2008 doi: 10.1111/j.1600-051X.2007.01195.x).

### SUMMARY OF THE INVENTION

In view of the problems in the prior art, the present invention provides in one aspect a method for cleaning a dental implant in a patient, comprising placing an isolation device around the dental implant to isolate the dental implant from other teeth of the patient, and air polishing the surface of the dental implant with β-tricalcium phosphate powder having a particle size of equal to or less than 350 μm for a period of no more than 90 seconds.

According to certain preferred embodiments of the present invention, the method comprising air polishing the surface of the dental implant with β-tricalcium phosphate powder having a particle size ranging from 100 μm to 200 μm for a period of no more than 50 seconds.

In certain embodiments of the present invention, the particle size ranges from 100 μm to 180 μm.

In certain embodiments of the present invention, the period is no more than 30 seconds. According to one embodiment, the period is about 20 seconds.

According to the present invention, the isolation device may comprise a flexible sleeve including a sleeve part and two extension parts extending from opposites sides of the sleeve part to define a space with the sleeve part, and an expanding fork adapted to fit in the flexible sleeve for expanding the sleeve part.

In another aspect, the present invention provides an isolation device for use in air powder abrasive treatment of a dental implant, comprising a flexible sleeve including a sleeve part and two extension parts extending from opposites sides of the sleeve part to define a space with the sleeve part, and an expanding fork adapted to fit in the flexible sleeve for expanding the sleeve part.

In certain embodiments of the present invention, the expanding fork comprises a connecting part, and two prongs extending from opposite sides of the connecting part, each prong having a protruding part. Preferably, the protruding part has a through hole formed thereon.

Alternatively, the isolation device may comprise a sleeve body with an extension piece extending from a side of the sleeve body to a second end, the extension piece having a notch formed at the second end adapted to butt against the dental implant or a post thereof.

Without further elaboration, it is believed that a person of ordinary skill in the art may utilize the present invention to its broadest extent. Accordingly, it can be understood that the following description is provided only for the purposes of illustration and demonstration, and is not intended to limit the other disclosure in any way.

### BRIEF DESCRIPTION OF THE DRAWINGS

FIG. 1 is a perspective view of an isolation device according to one embodiment of the present invention.

FIG. 2 is a perspective view of a flexible sleeve of the isolation device as shown in FIG. 1.

FIG. 3 is a perspective view of an expansion fork of the isolation device as shown in FIG. 1.

FIG. 4 is another perspective view of the isolation device as shown in FIG. 1.

FIG. 5 is a perspective view of an isolation device according to another embodiment of the present invention.

FIG. 6 is another perspective view of the isolation device as shown in FIG. 5.

FIG. 7 shows the cleaning area rates by β-tricalcium phosphate powder with different particle sizes.

### DETAILED DESCRIPTION OF THE INVENTION

Unless otherwise specified, all technical and scientific terms used herein have the meanings as commonly known by those of ordinary skill in the art to which the present invention belongs.

The singular forms “a” and “an” as used herein, unless otherwise specified, refer to at least one (one or more) in quantity.

In one aspect, the present invention provides a method for cleaning a dental implant in a patient. The method comprises placing an isolation device around the dental implant to isolate the dental implant from other teeth of the patient, and air polishing the surface of the dental implant with β-tricalcium phosphate powder having a particle size of equal to or less than 350 μm for a period of no more than 90 seconds.

Preferably, the method comprising air polishing the surface of the dental implant with β-tricalcium phosphate powder having a particle size ranging from 100 μm to 200 μm for a period of no more than 50 seconds.

In certain embodiments of the present invention, the particle size ranges from 100 μm to 180 μm. In one specific embodiment, the particle size ranges from 105 μm to 177 μm.

In certain embodiments of the present invention, the period is no more than 30 seconds. According to one embodiment of the present invention, the period is about 20 seconds.

According to the present invention, the isolation device comprises a flexible sleeve and an expanding fork. The flexible sleeve includes a sleeve part and two extension parts. The two extension parts extend from opposites sides of the sleeve part to define a space with the sleeve part. The expanding fork is adapted to fit in the flexible sleeve for expanding the sleeve part.

In another aspect, the present invention provides an isolation device for use in air powder abrasive treatment of a dental implant, comprising a flexible sleeve including a sleeve part and two extension parts extending from opposites sides of the sleeve part to define a space with the sleeve part, and an expanding fork adapted to fit in the flexible sleeve for expanding the sleeve part.

In certain embodiments of the present invention, the expanding fork comprises a connecting part, and two prongs extending from opposite sides of the connecting part, each prong having a protruding part. Preferably, the protruding part has a through hole formed thereon.

Alternatively, the isolation device may comprise a sleeve body with an extension piece extending from a side of the sleeve body to a second end, the extension piece having a notch formed at the second end adapted to butt against the dental implant or a post thereof. According to the present invention, the sleeve body may have more than one (e.g., two) extension piece.

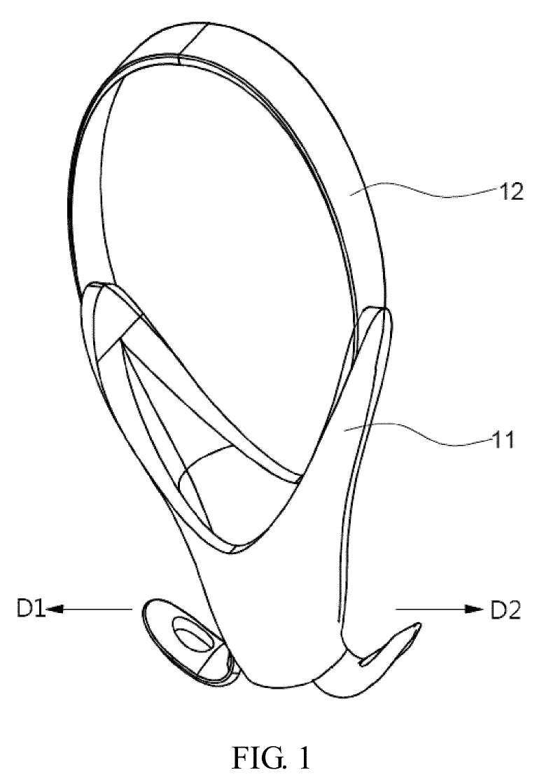

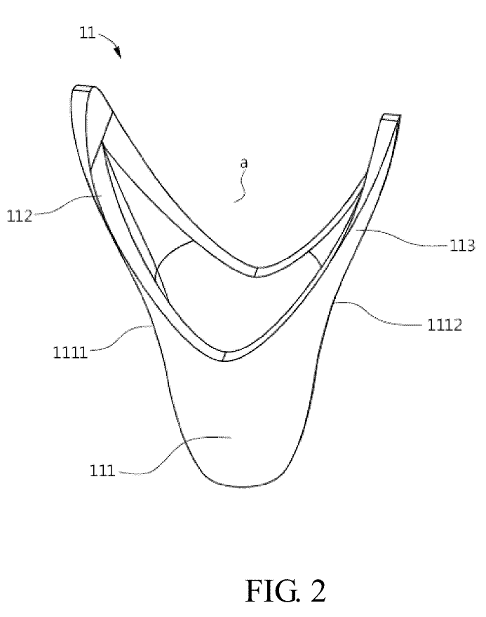

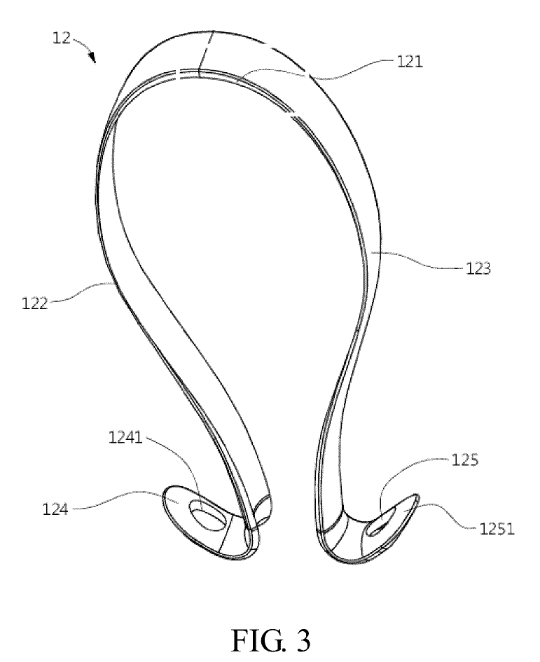

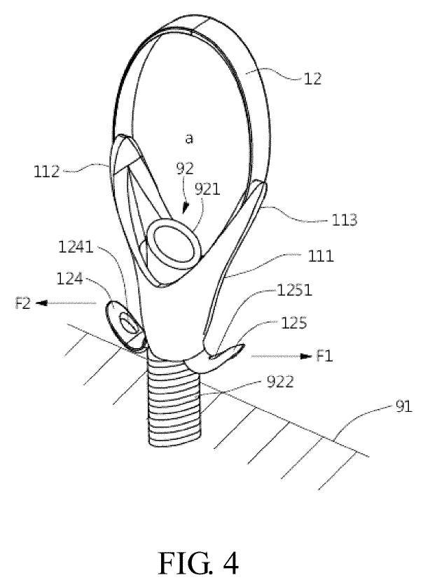

Referring to FIG. 1 through FIG. 4, provided is an isolation device comprising a flexible sleeve 11 and an expansion fork 12. The flexible sleeve 11 may be attached to a tooth implant 92 on a gum tissue 91. The flexible sleeve 11 includes a sleeve part 111 and two extension parts 112, 113. The two extension parts 112, 113 extend from opposites sides of the sleeve part 111 to define a space a with the sleeve part 111. The expansion fork 12 comprises a connecting part 121, and two prongs 122, 123 extending from opposite sides of the connecting part 121, each prong having a protruding part (124, 125). The expanding fork 12 is adapted to fit in the flexible sleeve 11 for expanding the sleeve part 111. The protruding parts 124, 125 may have a through hole (1241, 1251) formed thereon, and extend along directions D1 and D2, respectively, away from the connecting part 121.

The protruding parts 124, 125 may be subjected to force F2, F1 via the through holes 1241, 1251 along the directions D1, D2, respectively, so as to expand the sleeve part 111 to allow the tooth implant 92 to enter into the space a.

The present invention is further illustrated by the following examples, which are provided for the purpose of demonstration rather than limitation.

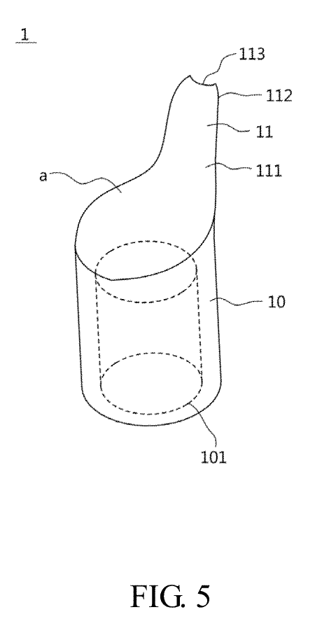

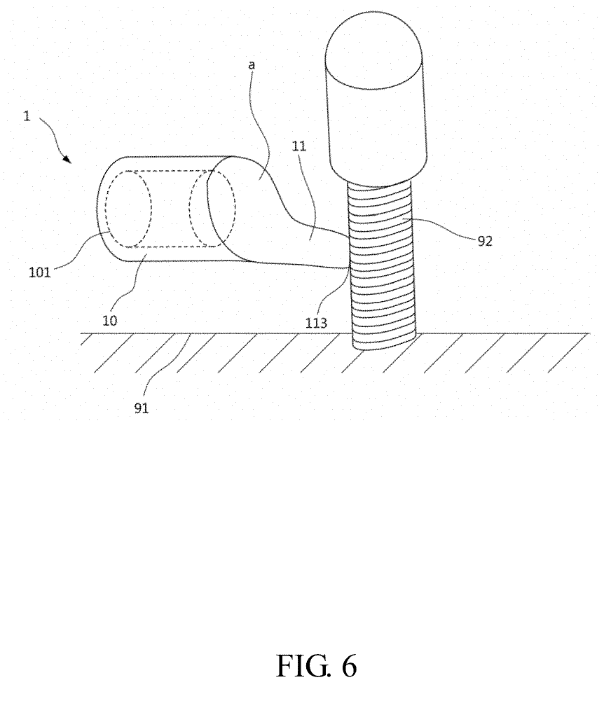

Referring to FIG. 5 and FIG. 6, shown is another isolation device 1. The isolation device 1 comprises a sleeve body 10 with an extension piece 11 extending from a side of the sleeve body 10, from a first end 111 to a second end 112. The sleeve body 10 has an accommodation space 101. The extension piece 11 has a notch 113 formed at the second end 112, the notch 113 being adapted to butt against a post 92 of a dental implant. A working space a is defined by the sleeve body 10 and the extension piece 11.

### EXAMPLES

### Example 1: Comparison of Different Particle Sizes of β-Tricalcium Phosphate Powder in Cleaning Dental Implants

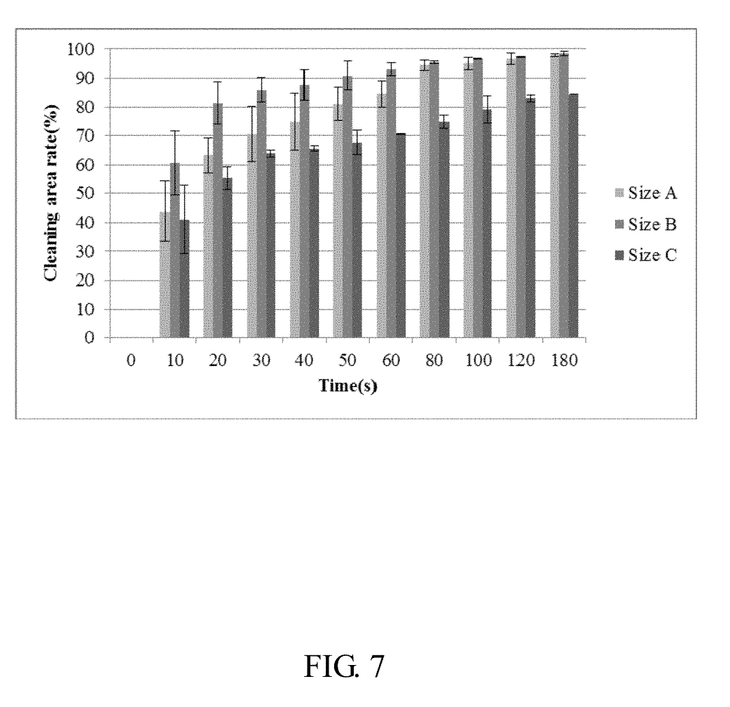

Dental implants (lengths: 9.5 mm Ø: 3.5 mm) were immersed in the permanent non-covering ink (Staedler permanent Lumocolor) by using a dip coating control system (ANKYLOS, DENTSPLY Implants, Germany) in 10 seconds. Place the dental implants in the hood for 48 hours. 1-2 mm β-TCP (beta-tricalcium phosphate) particles were grinded and sieved into three different particle sizes, 53-105 μm (size A), 105-177 μm (size B) and 250-350 μm (size C). Stick the hydrocolloid dressing (4×10 mm and 43×15.5 mm) to the L-shaped fixture. Remove a dental implant from the hood after 48 hours, and place the implant in the L-shaped fixture (3 mm from the outlet) and fix it with metal plates, screws and nuts. Clean the implant for 10 seconds by using an air polisher system (Prophy-APII), and cleaned dental implant is photographed by camera (Canon EOS 60D). The dental implant was cleaned and photographed after cleaning at 20, 30, 40, 50, 60, 80, 100, 120, and 180 seconds. At the end of the experiment, remove the water stains on the surface of the dental implant and change the clean surface (release the metal plate and flip the dental implant in parallel). Then, use Adobe Photoshop CS6 to process images and Visual Studio 2013-Open CV to calculate cleaning area rates (the area that can be cleaned by β-TCP). The results are shown in FIG. 7 and Table 1 below.

TABLE 1
Amount of powder used and time required to achieve a cleaning
rate of higher than 80%

| | | |
|---|---|---|
| Size A | Powder used (g/min) | 0.0176 |
| | Time to achieve a cleaning rate of | 50 |
| | higher than 80% (s) | |
| Size B | Powder used (g/min) | 0.0059 |
| | Time to achieve a cleaning rate of | 20 |
| | higher than 80% (s) | |
| Size C | Powder used (g/min) | 0.0043 |
| | Time to achieve a cleaning rate of | 120 |
| | higher than 80% (s) | |

Surprisingly, size B (105-177 μm) β-TCP achieves a cleaning rate of higher than 80% in 20 seconds which is significantly shorter than the time needed by using size A (53-105 μm) or size C (250-350 μm) β-TCP, and is beneficial in preventing damaging tissues. Further, as compared to size A β-TCP which has an acceptable cleaning efficiency, the amount of powder used in the air polishing for size B β-TCP is significantly lower.

Although the present invention has been described as above, the disclosure is not used to limit the scope, order of implementation, or the materials and process methods used. Various modifications and changes made to the present invention are covered by the spirit and scope of the present invention.

What is claimed is:

1. A method for cleaning a dental implant in a patient, comprising:
placing an isolation device around or against the dental implant to isolate the dental implant from other teeth of the patient; and
air polishing the surface of the dental implant with β-tricalcium phosphate powder having a particle size of equal to or less than 350 μm for a period of no more than 90 seconds.

2. The method of claim 1, comprising air polishing the surface of the dental implant with β-tricalcium phosphate powder having a particle size ranging from 100 μm to 200 μm for a period of no more than 50 seconds.

3. The method of claim 2, wherein the particle size ranges from 100 μm to 180 μm.

4. The method of claim 2, wherein the period is no more than 30 seconds.

5. The method of claim 4, wherein the period is about 20 seconds.

6. The method of claim 1, wherein the isolation device comprises:
a flexible sleeve including a sleeve part and two extension parts extending from opposites sides of the sleeve part to define a space with the sleeve part; and
an expanding fork adapted to fit in the flexible sleeve for expanding the sleeve part.

7. The method of claim 2, wherein the isolation device comprises:
a flexible sleeve including a sleeve part and two extension parts extending from opposites sides of the sleeve part to define a space with the sleeve part; and
an expanding fork adapted to fit in the flexible sleeve for expanding the sleeve part.

8. The method of claim 1, wherein the isolation device comprises a sleeve body with an extension piece extending from a side of the sleeve body to a second end, the extension piece having a notch formed at the first end adapted to butt against the dental implant or a post thereof.

9. An isolation device for use in air powder abrasive treatment of a dental implant, comprising:
a flexible sleeve including a sleeve part and two extension parts extending from opposites sides of the sleeve part to define a space with the sleeve part; and
an expanding fork adapted to fit in the flexible sleeve for expanding the sleeve part.

10. The isolation device of claim 8, wherein the expanding fork comprises a connecting part, and two prongs extending from opposite sides of the connecting part, each prong having a protruding part.

11. The isolation device of claim 9, wherein the protruding part has a through hole.
